(19) United States
(12) Patent Application Publication
(10) Pub. No.: US 2024/0350275 A1
(43) Pub. Date: Oct. 24, 2024

(54) AUGMENTED GLENOID IMPLANT AND METHOD OF INSTALLING THE SAME DURING AN ORTHOPAEDIC SHOULDER PROCEDURE

(71) Applicant: DePuy Ireland Unlimited Company, Ringaskiddy (IE)

(72) Inventors: Brian C. HODOREK, Winona Lake, IN (US); Alec CUNNINGHAM, Fort Wayne, IN (US); Colton D. MYERS, Warsaw, IN (US); Russell M. PARROTT, Winona Lake, IN (US); J. Michael WIATER, Beverly Hills, MI (US); Anand M. MURTHI, Baltimore, MD (US); Matthew J. SMITH, Columbia, MO (US); Derek J. CUFF, Venice, FL (US); Andrew JAWA, Cambridge, MA (US); Luke AUSTIN, Haddonfield, NJ (US); Matthew J. PURDY, Winona Lake, IN (US)

(21) Appl. No.: 18/636,635

(22) Filed: Apr. 16, 2024

Related U.S. Application Data
(60) Provisional application No. 63/497,702, filed on Apr. 21, 2023.

(51) Int. Cl. A61F 2/40; A61B 17/16; A61F 2/30

(52) U.S. Cl. CPC A61F 2/4081; A61B 17/1684; A61F 2/30734; A61F 2/30749; A61F 2002/30326; A61F 2002/30507; A61F 2002/30736

(57) ABSTRACT

A glenoid implant includes a baseplate, a pair of bone screws, and an augmentation member. The baseplate includes a plurality of screw holes with the pair of bone screws being positioned in two of the screw holes and the augmentation member being positioned in a third screw hole. The augmentation member includes a non-threaded post configured to abut the bottom of a bone defect. A method of installing a glenoid implant during an orthopaedic surgical procedure is also disclosed.

The present application claims priority under 35 U.S.C. § 119 to U.S. Patent application Ser. No. 63/497,702, filed Apr. 21, 2023, and having the title “ORTHOPEDIC IMPLANT SYSTEMS WITH AUGMENTATION DEVICE AND METHODS OF USE,” which is herein incorporated by reference in its entirety.

### CROSS-REFERENCE TO RELATED APPLICATION

Cross reference is made to copending U.S. patent application Ser. No. ______ entitled “GLENOID IMPLANT AND METHOD OF INSTALLING THE SAME DURING AN ORTHOPAEDIC SHOULDER PROCEDURE” (Attorney Docket No. 265280-407399, DEP7158USNP1), which is assigned to the same assignee as the present application, is filed concurrently herewith, and is hereby incorporated by reference.

### TECHNICAL FIELD

The present invention relates generally to orthopaedic surgery, implant systems used for replacing an articulation surface in a joint, such as, for example, shoulder prostheses. More specifically, but not exclusively, the present invention relates to glenoid implants for shoulder arthroplasties having augmentation devices to provide stability to a glenoid baseplate when implanted over a glenoid deficiency, as well as methods for using the same.

### BACKGROUND

Shoulder replacement, also known as shoulder arthroplasty or glenohumeral arthroplasty, is a surgical procedure in which all or part of the glenohumeral joint is replaced by a prosthetic implant. Such joint replacement surgery generally is conducted to relieve arthritis pain or fix severe physical joint damage.

In a total shoulder replacement procedure, a humeral prosthesis is used to replace the natural head of the patient's humerus. The humeral prosthesis typically includes an elongated stem component that is implanted into the intramedullary canal of the patient's humerus and a hemispherically-shaped prosthetic head component that is secured to the stem component. In such a total shoulder replacement procedure, the natural glenoid surface of the scapula is resurfaced or otherwise replaced with a glenoid component that provides a bearing surface upon which the prosthetic head component of the humeral prosthesis articulates.

However, in some cases the patient's natural shoulder, including its soft tissue, has degenerated to a severe degree of joint instability and pain. In many such cases, it may be necessary to change the mechanics of the shoulder. Reverse shoulder implants are used to do so. As its name suggests, a reverse shoulder implant reverses the anatomy, or structure, of the healthy shoulder. In particular, a reverse shoulder implant is designed such that the prosthetic head (i.e., the “ball” in the ball-and-socket joint) known as a glenosphere component is secured to the patient's scapula, with the corresponding concave bearing (i.e., the “socket” in the ball-and-socket joint) known as a humeral cup being secured to the patient's humerus. Such a reverse configuration allows the patient's deltoid muscle, which is one of the larger and stronger shoulder muscles, to raise the arm.

During a shoulder replacement procedure, a surgeon may encounter bone defects, such as voids or glenoid deficiencies, in various areas of a glenoid cavity during anatomic or reverse arthroplasty. To account for such bone defects, various augmented glenoid baseplates exist that have predetermined shapes on their backsides. The predetermined shapes are designed to more closely fit various types of glenoid defects. Such augmented baseplates require specific preparation of the glenoid cavity to create the specific angles and depths required to receive the unique geometry of the augmented baseplates. Moreover, a large inventory of instruments and augmented implants with various backside shapes may need to be brought into the operating room to accommodate the numerous glenoid bone defects the surgeon may encounter since the surgeon may not be able to determine which augmented baseplate is required until the patient's glenoid is surgically exposed.

### SUMMARY

According to one aspect of the disclosure, a glenoid implant for implantation into a glenoid of a patient during an orthopaedic surgical procedure includes a baseplate having a plate with a top surface and a bottom surface. The plate includes a central opening extending into the plate from the top surface, and a plurality of screw holes extending from the top surface to the bottom surface of the plate. The plurality of screw holes are positioned circumferentially around the plate. The glenoid implant also includes a shaft coupled to and extending downwardly away from a center of the bottom surface of the plate. The shaft has a number of threads formed in an outer surface thereof. The glenoid implant also includes a first bone screw extending downwardly away from the bottom surface of the plate. The first bone screw has a head positioned in a first screw hole of the plurality of screw holes, and an elongated threaded shank extending away from its threaded head. The glenoid implant also includes a second bone screw extending downwardly away from the bottom surface of the plate. The second bone screw has a head positioned in a second screw hole of the plurality of screw holes, and an elongated threaded shank extending away from its threaded head. Yet further, the glenoid implant includes an augmentation member extending downwardly away from the bottom surface of the plate. The augmentation member has a threaded head positioned in a third screw hole of the plurality of screw holes, and an elongated non-threaded post extending away from its threaded head.

In an embodiment, the elongated non-threaded post of the augmentation member has a porous structure disposed thereon.

In an embodiment, the elongated non-threaded post of the augmentation member is shorter in length than the elongated threaded shanks of the first and second bone screws.

The plate of the baseplate may further include a peripheral ring extending downwardly away from, and circumferentially around, the bottom surface of the plate. The peripheral ring has an interior surface and an opposite exterior surface, with the exterior surface of the peripheral ring having a number of threads formed therein.

The peripheral ring may include a plurality of separate arcuate-shaped ring portions separated from one another by a gap.

In an embodiment, the shaft is integral with the plate.

The glenoid implant may also include a locking screw and a glenosphere. In such an embodiment, the central opening of the plate may have a number of threads formed therein. Likewise, the glenosphere may have a number of threads formed therein. The locking screw has a first number of threads configured to engage the threads of the central opening and a second number of threads configured to engage the threads of the glenosphere.

In another aspect, a glenoid implant for implantation into a glenoid of a patient during an orthopaedic surgical procedure includes a baseplate having a plate with a top surface and a bottom surface. The plate includes a central opening extending into the plate from the top surface, and a plurality of screw holes extending from the top surface to the bottom surface of the plate. The plurality of screw holes are positioned circumferentially around the plate. The glenoid implant also includes a shaft coupled to and extending downwardly away from a center of the bottom surface of the plate. The shaft has a number of threads formed in an outer surface thereof. The glenoid implant also includes a pair of bone screws extending downwardly away from the bottom surface of the plate. Each of the pair of bone screws has a head positioned in one of the plurality of screw holes, and an elongated threaded shank extending away from its threaded head. The glenoid implant also includes an augmentation member extending downwardly away from the bottom surface of the plate. The augmentation member has a threaded head positioned in one of the plurality of screw holes, and an elongated non-threaded post extending away from its threaded head. The glenoid implant also includes a glenosphere having a number of threads formed therein, and a locking screw. The locking screw has a first number of threads configured to engage the threads of the central opening and a second number of threads configured to engage the threads of the glenosphere.

In an embodiment, the elongated non-threaded post of the augmentation member has a porous structure disposed thereon.

In an embodiment, the elongated non-threaded post of the augmentation member is shorter in length than the elongated threaded shanks of the pair of bone screws.

The plate of the baseplate may further include a peripheral ring extending downwardly away from, and circumferentially around, the bottom surface of the plate. The peripheral ring has an interior surface and an opposite exterior surface, with the exterior surface of the peripheral ring having a number of threads formed therein.

The peripheral ring may include a plurality of separate arcuate-shaped ring portions separated from one another by a gap.

In an embodiment, the shaft is integral with the plate.

According to another aspect, a method of surgically implanting a glenoid implant into a glenoid of a patient's shoulder during performance of an orthopaedic surgical procedure includes surgically exposing an outer surface of the glenoid. The outer surface of the glenoid has a bone defect. Thereafter, the outer surface of the glenoid is reamed so as to form (i) a circular surface in the outer surface of the glenoid, (ii) a central bore hole that is concentric with the portion of the circular surface, and (iii) a circular groove that is concentric with the central bore hole. A baseplate of the glenoid implant is inserted into the central bore hole and circular groove. The baseplate has a plurality of screw holes. A first screw hole of the plurality of screw holes of the baseplate is aligned over the bone defect. An augmentation member is inserted through the first screw hole such that a bottom surface of a non-threaded post of the augmentation member contacts a bottom surface of the bone defect.

In an embodiment, a first bone screw is inserted through a second screw hole of the plurality of screw holes of the baseplate such that a threaded shank of the first bone screw is threaded into the glenoid, and a second bone screw is inserted through a third screw hole of the plurality of screw holes of the baseplate such that a threaded shank of the second bone screw is threaded into the glenoid.

A locking screw of the glenoid implant may be into a central opening in a top surface of the baseplate, and a glenosphere of the glenoid implant may be coupled to the locking screw.

The glenoid around the bone defect may be reamed prior to insertion of the augmentation member.

### BRIEF DESCRIPTION OF THE DRAWINGS

The detailed description particularly refers to the following figures, in which:

FIG. 1 is a side perspective view of a glenoid implant system;

FIG. 2 is a cross-sectional view of the glenoid implant system of FIG. 1 taken along line 2-2 in FIG. 1, as viewed in the direction of the arrows;

FIG. 3 is an exploded perspective view of the glenoid implant system of FIG. 1;

FIG. 4 is another exploded perspective view of the glenoid implant system of FIG. 1;

FIG. 5 is a perspective view of the baseplate of the glenoid implant system of FIG. 1;

FIG. 6 is another perspective view of the baseplate of FIG. 5;

FIG. 7 is a side view of the baseplate of FIG. 5;

FIG. 8 is another side view of the baseplate of FIG. 5;

FIG. 9 is an end view of the baseplate of FIG. 5;

FIG. 10 is another end view of the baseplate of FIG. 5;

FIG. 11 is a top view of the baseplate of FIG. 5;

FIG. 12 is a bottom view of the baseplate of FIG. 5;

FIG. 13 is an exploded perspective view of the baseplate of FIG. 5;

FIG. 14 is another exploded perspective view of the baseplate of FIG. 5;

FIG. 15 is an exploded side view of the baseplate of FIG. 5;

FIG. 16 is an exploded perspective view of another glenoid implant system;

FIG. 17 is another exploded perspective view of the implant system of FIG. 16;

FIG. 18 is an exploded perspective view of the baseplate of the implant system of FIG. 16;

FIG. 19 is another exploded perspective view of the baseplate of FIG. 18;

FIG. 20 is an exploded side view of the baseplate of FIG. 18;

FIG. 21 is another exploded side view of the baseplate of FIG. 18;

FIG. 22 is an exploded end view of the baseplate of FIG. 18;

FIG. 23 is another exploded end view of the baseplate of FIG. 18;

FIG. 24 is an exploded top view of the baseplate of FIG. 18;

FIG. 25 is an exploded bottom view of the baseplate of FIG. 18;

FIG. 26 is an exploded perspective view of another glenoid implant system;

FIG. 27 is another exploded perspective view of the implant system of FIG. 26;

FIG. 28 is an exploded perspective view of the baseplate of the implant system of FIG. 26;

FIG. 29 is another exploded perspective view of the baseplate of FIG. 28;

FIG. 30 is an exploded side view of the baseplate of FIG. 28;

FIG. 31 is another exploded side view of the baseplate of FIG. 28;

FIG. 32 is an exploded end view of the baseplate of FIG. 28;

FIG. 33 is another exploded end view of the baseplate of FIG. 28;

FIG. 34 is an exploded top view of the baseplate of FIG. 28;

FIG. 35 is an exploded bottom view of the baseplate of FIG. 28;

FIG. 36 is a perspective view of the assembled baseplate of FIG. 28; and

FIG. 37 is a cross-sectional view of the assembled glenoid implant system of FIG. 26.

### DETAILED DESCRIPTION OF THE DRAWINGS

Generally stated, disclosed herein are implant systems and methods of making the same. Further, surgical methods for using the implant systems are discussed.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in the first figure of each embodiment.

Similarly, positions or directions may be used herein with reference to anatomical structures or surfaces. For example, as the current implant systems (or implants), devices, systems and methods are described herein with reference to use with the bones of the shoulder, the bones of the shoulder and upper arm may be used to describe the surfaces, positions, directions or orientations of the implant systems, devices, systems and methods. Further, the implant systems, devices, systems and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implant systems, devices, systems and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implant systems, devices, systems and methods, and the aspects, components, features and the like thereof, described herein with respect to the right shoulder may be mirrored so that they likewise function with the left shoulder and vice versa. Further, the implant systems, devices, systems and methods, and the aspects, components, features and the like thereof, disclosed herein are described with respect to the shoulder for brevity purposes, but it should be understood that the implant systems, devices, systems and methods may be used with other bones of the body having similar structures, for example the lower extremity, and more specifically, with the bones of the ankle, foot, and leg.

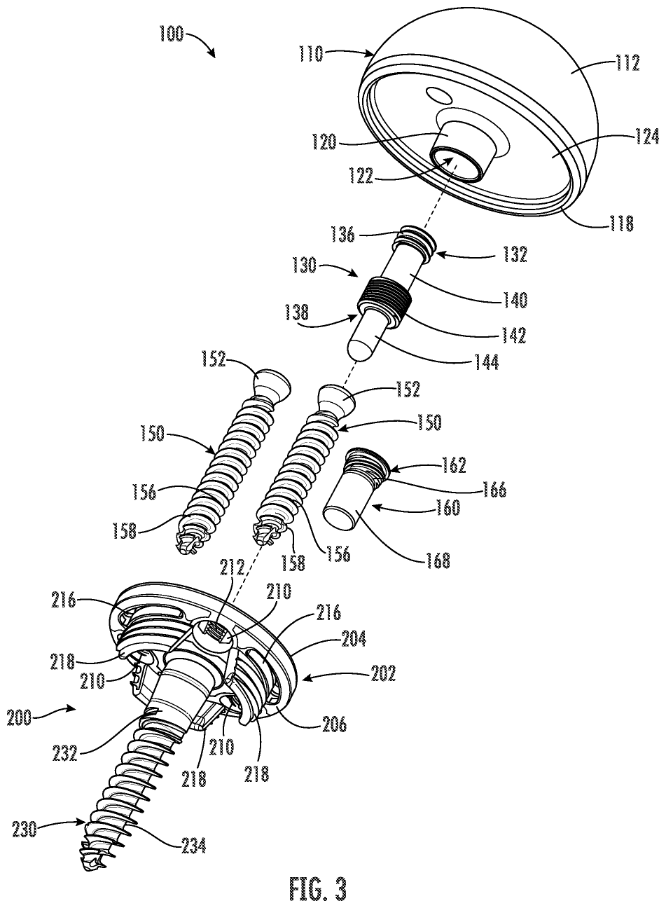

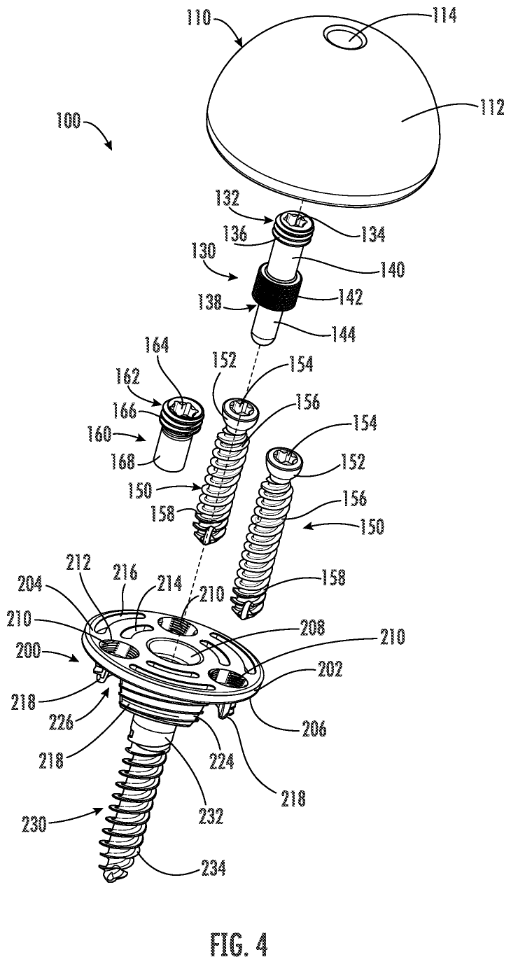

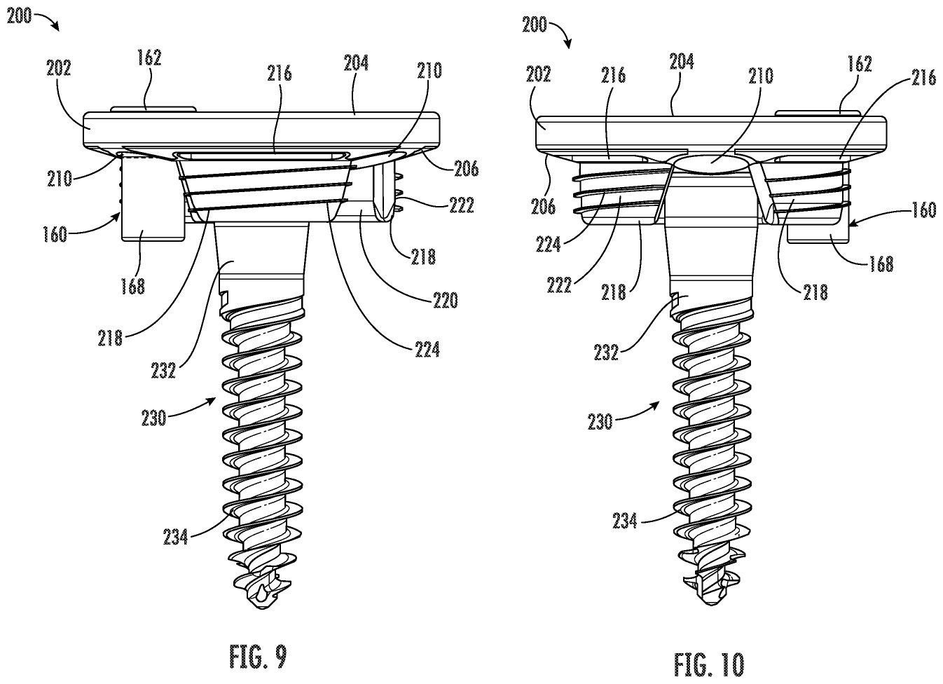

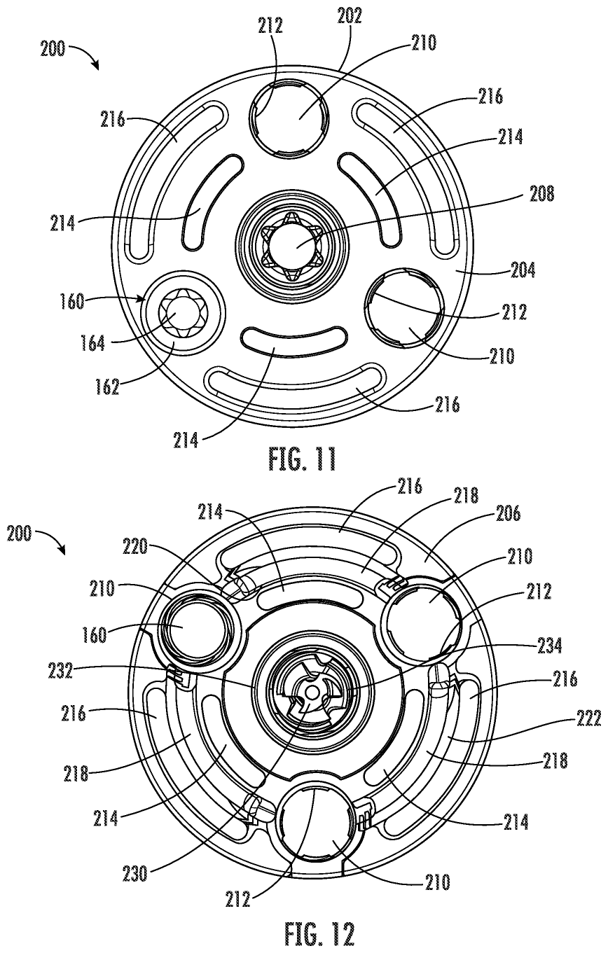

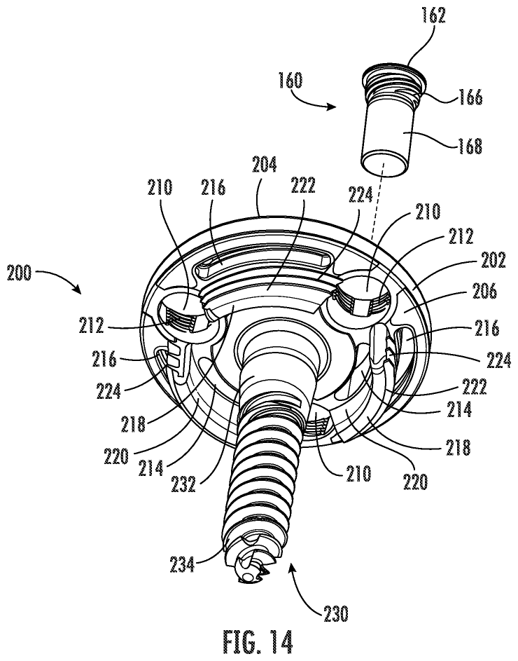

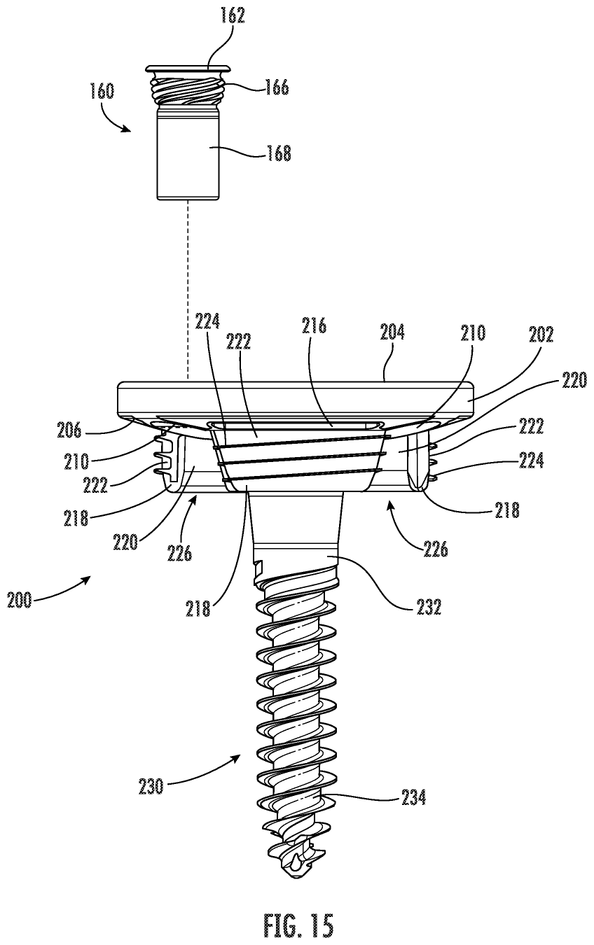

Referring to the drawings, wherein like reference numerals are used to indicate like or analogous components throughout the several views, and with particular reference to FIGS. 1-15, there is illustrated an embodiment of a glenoid implant or reverse glenoid implant 100. The implant 100 may include a baseplate 200, a locking screw 130, and at least one augmentation member 160. The locking screw 130 and at least one augmentation member 160 are received within the baseplate 200. The implant 100 may also include one or more peripheral screws or screws 150 for insertion through the baseplate 200. The implant 100 may further include a glenosphere 110 coupled to the baseplate 200 by the locking screw 130.

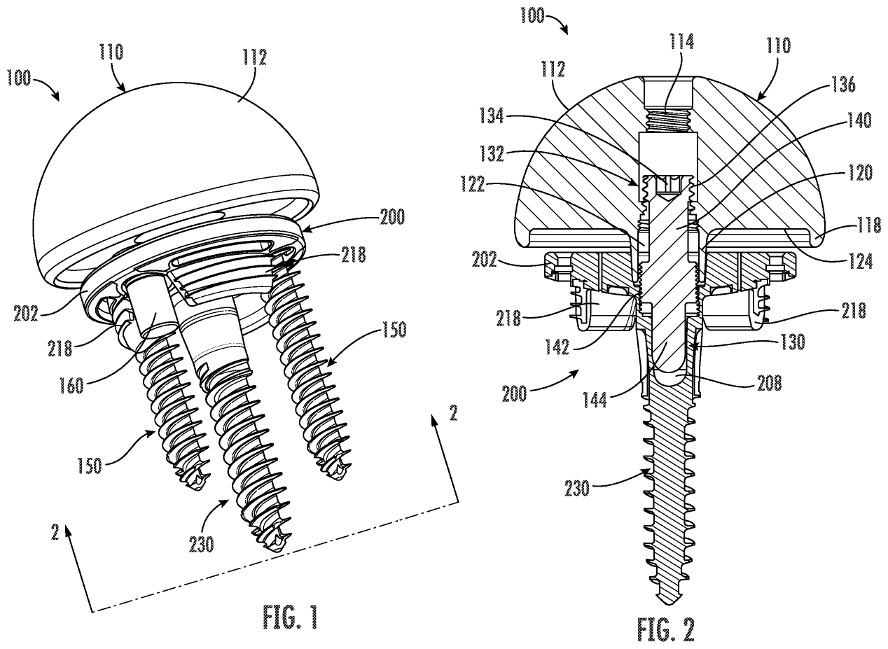

As shown in FIGS. 1-4, the glenosphere 110 may include an articulating surface 112 on a top side and an engagement surface 116 on a bottom side. The articulating surface 112 may include an opening 114 extending into the glenosphere 110 from the articulating surface 112 toward the engagement surface 116. The opening 114 may be, for example, threaded or partially threaded. The engagement surface 116 receives a portion of the baseplate 200. The engagement surface 116 includes a rim 118 positioned on the exterior surface of the engagement surface 116 to form a recessed region 124 for receiving the baseplate 200. The engagement surface 116 also includes a protrusion 120 extending away from the recessed region 124 at or near a midpoint of the glenosphere 110. The glenosphere 110 also includes a through hole 122 extending into the glenosphere 110 from the engagement surface 116 through the protrusion 120 and engaging the opening 114. The through hole 122 may include at least one threaded portion, as seen in FIG. 2. The through hole 122 may also include, for example, an integral taper along a portion of the length of the interior surface of the through hole 122.

Referring now to FIGS. 2-4, the locking screw 130 is shown. The locking screw 130 includes a head 132 at a first end and a shaft 138 extending away from the head 132 to the second end. The head 132 includes an engagement opening 134 extending into the first end of the head 132. The engagement opening 134 is configured or sized and shaped to receive a tool for rotating the locking screw 130. The locking screw 130 may also include threads 136 extending around at least a portion of the exterior surface of the head 132. The threads 136 may be configured or sized and shaped to engage threads in the through hole 122.

The shaft 138 may include a first non-threaded portion 140, a threaded portion 142, and a second non-threaded portion 144, as shown in FIGS. 2-4. The first non-threaded portion 140 couples to and extends from the head 132 toward the second end of the locking screw 130. The threaded portion 142 is positioned between the first non-threaded portion 140 and the second non-threaded portion 144. The second non-threaded portion 144 extends from the second end of the locking screw 130 to the threaded portion 142. The threaded portion 142 may be, for example, positioned near a midpoint of the shaft 138. The threaded portion 142 may be, for example, positioned between the first non-threaded portion 140 and the second non-threaded portion 144. The first and second non-threaded portions 140, 144 may be, for example, the same or different lengths. The threaded portion 142 may, for example, have a length the same size or smaller than the first and second non-threaded portions 140, 144.

With continued reference to FIGS. 1-4, the screws 150 include a head 152 and a shank 156 coupled to and extending from the head 152. The head 152 may include an opening 154 extending into the first end of the head 152. The opening 154 may be configured or sized and shaped to receive a tool, such as a screwdriver, for rotating the screws 150. The shank 156 may include threads 158 extending along the length of the shank 156 for engaging bone.

Referring now to FIGS. 1-15, the at least one augmentation member 160 and the baseplate 200 are shown. The at least one augmentation member 160 is received within the baseplate 200. The augmentation member 160 includes a head 162 and a post or void filler 168 coupled to and extending away from the head 162. The head 162 includes an opening 164 extending into the first end of the head 162. The opening 164 may be, for example, configured or sized and shaped to receive a tool for rotating the augmentation member 160. The head 162 may also include threads 166 positioned around the exterior surface of the head 162. The post 168 may have various lengths and widths to correspond to the voids in a patient's bone. The post 168 may have, for example, a cylindrical shape or another shape, such as a square or rectangular cross-sectional shape. In addition, the post 168 may have, for example, a shape that conforms to the shape of the through hole it is extended through. The post 168 may be sized to enable the augmentation member 160 to traverse or fill a defect in a bone. As shown in, for example, FIGS. 1, 3-5, 7-10, and 13-15, unlike the threaded shanks 156 of the bone screws 150, the post 168 of the augmentation member 160 is non-threaded. The post 168 may have, for example, a porous structure extending from the threads 166 to the second end of the post 168. The porous structure may be, for example, 3D printed for allowing for bone ingrowth from the bone surrounding the void where the augmentation member 160 is positioned within. The porous structure may be, for example, a porous metal structure or porous coating.

The baseplate 200 may have a plate 202 and a shaft 230 coupled to and extending away from a bottom surface 206 of the baseplate 200, as shown in FIGS. 1-15. The plate 202 may have a top surface 204 and a bottom surface 206 opposite the top surface 204. The plate 202 may have, for example, a cylindrical shape with a circular top surface 204 and a circular bottom surface 206. The at least a portion of the bottom surface 206 may have, for example, a porous structure. The porous structure may be, for example, 3D printed for allowing for bone ingrowth from the bone surrounding the baseplate 200. The porous structure may be, for example, a porous metal structure or porous coating. The plate 202 may also include a central opening 208 extending from the top surface 204 through the plate 202 and into a portion of the shaft 230. The central opening 208 may be positioned, for example, in a central region of the plate 202. The central opening 208 may be, for example, configured or sized and shaped to receive a portion of the locking screw 130. The central opening 208 may include threads along at least a portion of the central opening 208. The threads in the central opening 208 may be, for example, configured or sized and shaped to correspond to the threads of the threaded portion 142 of the locking screw 130. The central opening 208 is also configured or sized and shaped to receive at least a portion of the protrusion 120. Thus, the central opening 208 may have, at least one diameter along its length, as shown in FIG. 2. The central opening 208 may have, for example, three diameters, a first diameter for receiving the protrusion 120, a second diameter for receiving the threaded portion 142 of the locking screw 130, and a third diameter for receiving the second non-threaded portion 144 of the locking screw 130.

The baseplate 200 may also include screw holes or holes 210, for example, three screw holes 210 equally spaced around the circumference of the plate 202. The screw holes 210 may extend through the plate 202 from the top surface 204 to the bottom surface 206. The screw holes 210 may have, for example, threads 212 along at least a portion of the interior surface of the screw holes 210. The screw holes 210 are configured or sized and shaped to receive a screw 150 for anchoring the baseplate 200 to a bone. The head 152 of the screw 150 may be seated within the screw hole 210 of the baseplate 200. The plate 202 may further include at least one relief or arcuate slot 214, 216 extending through the plate 202 from the top surface 204 to the bottom surface 206. The at least one relief 214, 216 may include at least one first or interior relief 214 and at least one second or exterior relief 216. The at least one first relief 214 may be, for example, three first reliefs 214 positioned circumferentially around the central opening 208 between the screw holes 210. The at least one second relief 216 may be, for example, three second reliefs 216 positioned circumferentially around the plate 202 between the screw holes 210. The first reliefs 214 are spaced apart from the second reliefs 216. The second reliefs 216 may be positioned near an exterior surface of the plate 202.

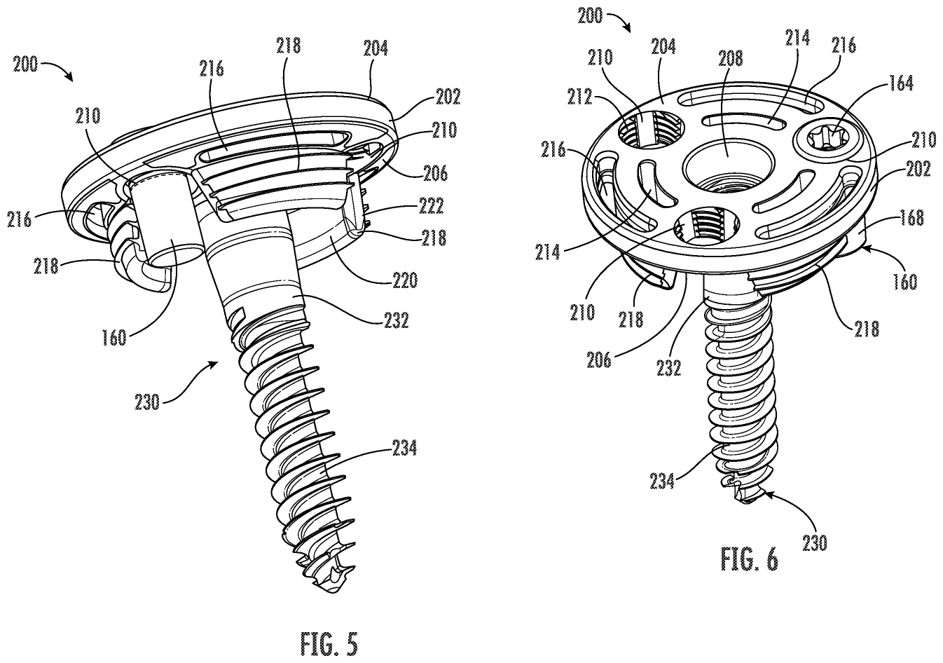

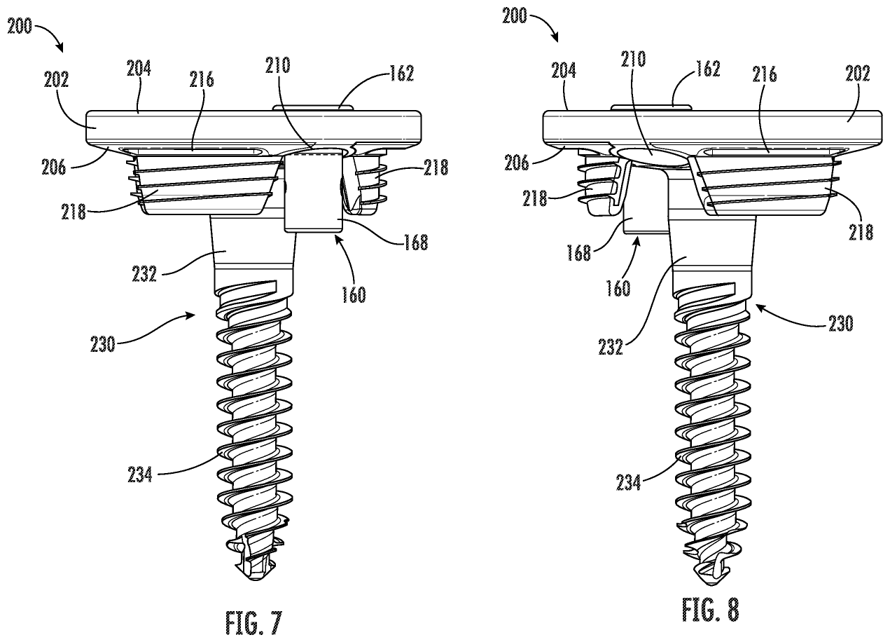

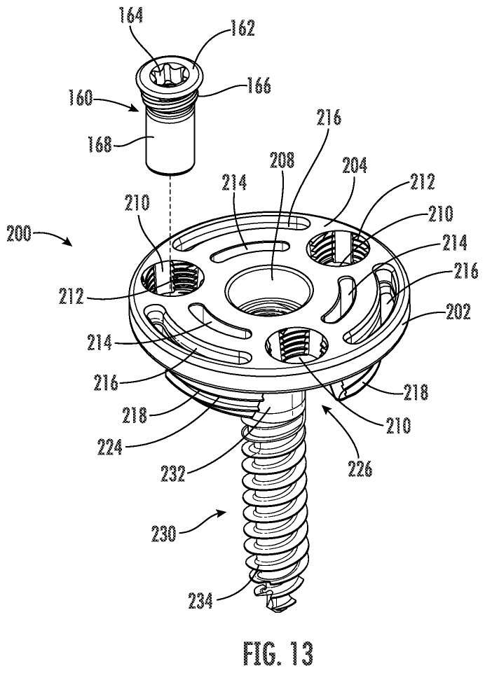

The plate 202 may include a peripheral ring illustratively in the form of, for example, at least one peripheral ring portion 218 extending away from the bottom surface 206. As shown in FIGS. 5 and 12, the peripheral ring may take the form of, for example, three arcuate-shaped peripheral ring portions 218 positioned circumferentially around the bottom surface 206. The peripheral ring portions 218 may be, for example, the same or different sizes. The peripheral ring portions 218 may be, for example, separated by a gap or space 226. The peripheral ring portions 218 may each include an interior surface 220 and an exterior surface 222. The interior surface 220 may be, for example, a uniform surface, while the exterior surface 222 may have, for example, threads 224 extending around the exterior surface 222. As can be seen in FIGS. 7-10, the threads 224 formed in the exterior surface 222 of the peripheral ring have a common thread pitch with the threads formed in the threaded portion 234 of the shaft 230. The interior and exterior surfaces 220, 222 may have, for example, a porous structure. The porous structure may be, for example, 3D printed. The at least one peripheral ring portion 218 may extend away from the bottom surface 206 of the plate 202 between the first reliefs 214 and the second reliefs 216. The peripheral ring portions 218 may be inserted to engage a prepared glenoid cavity. The peripheral ring portions 218 may be, for example, arcuate and follow the same profile as the first and second reliefs or slots 214, 216. The peripheral ring portions 218 may be, for example, positioned adjacent to the first and second slots 214, 216. In addition, the screw holes 210 in the plate 202 may be positioned between the peripheral ring portions 218 where the spaces 226 are positioned.

The shaft 230 of the baseplate 200 may have a non-threaded portion 232 and a threaded portion 234. The non-threaded portion 232 may be coupled to and extend from the bottom surface 206 of the plate 202. The threaded portion 234 may extend from the second end of the shaft 230 to the non-threaded portion 232. The shaft 230 may extend away from the bottom surface 206 of the plate 202 near a midpoint of the plate 202. The shaft 230 may be positioned opposite the central opening 208 extending into the top surface 204, through the plate 202, and into a portion of the shaft 230. The central opening 208 may extend into at least a portion of the non-threaded portion 232. The shaft 230 may be, for example, longer than the at least one peripheral ring portions 218. The shaft 230 may extend away from the bottom surface 206 of the plate 202 between the at least one peripheral ring portions 218. The non-threaded portion 232 may be a porous structure, which may be, for example, 3D printed. The porous structure of the non-threaded portion 232 may assist with bone ingrowth.

The plate 202 and shaft 230 are integrally connected in the embodiments of FIGS. 1-15, while the locking screw 130 and baseplate 200 are separate pieces. It is also contemplated that the plate 202 and shaft 230 could be separate pieces.

The implant 100 may be assembled by inserting and coupling the locking screw 130 into the central opening 208. The augmentation member 160 may then be inserted through into the screw holes 210 through the plate 202. The screws 150 may also be inserted through the screw holes 210 through the plate 202. Finally, the glenosphere 110 may be coupled to the head 132 of the locking screw 130. Alternatively, the glenosphere 110 may have been pre-assembled with the locking screw 130 before the locking screw 130 was inserted into the central opening 208.

A surgical method for preparing a bone and inserting the implant 100 into the bone is disclosed. The method includes preparing the glenoid cavity of a scapula for receiving the implant 100. A bone reamer (not shown) may be used to prepare a top or outer surface of the glenoid cavity that includes a bone defect, such as a void. The outer surface of the glenoid cavity is surgically exposed using a bone reamer to remove a portion of a circular surface into the outer surface of the glenoid cavity. If a bone defect is present, then the portion of the circular surface may not be a completely circular surface due to bone loss caused by the bone defect.

Next, the bone reamer may be used to drill a central bore hole in the glenoid cavity that is concentric with the portion of the circular surface for receiving the shaft 230 of the baseplate 200. Then, the bone reamer may cut a portion of a circular groove into the glenoid cavity bone that is concentric with the center bore hole. A plurality of cutting pegs of the bone reamer may be used to cut a portion of the circular groove. The portion of the circular groove may or may not be a complete circular groove for a full 360 degree groove due to pre-existing bone loss. The bone reamer may, for example, ream the portion of the circular surface, drill the center bore hole, and cut the portion of the circular groove substantially simultaneously. Alternatively, the portion of the circular surface may be reamed first, then the center bore hole may be drilled, and finally the portion of the circular groove may be cut into the glenoid cavity.

Once the glenoid cavity is prepared, the baseplate 200 of the implant 100 may be inserted into the prepared glenoid cavity with an insertion tool (not shown). The shaft 230 may be aligned with and inserted into the center bore hole by screwing the baseplate 200 into the glenoid cavity. As the baseplate 200 is inserted into the glenoid cavity, the peripheral ring portions 218 are inserted into the portion of the circular groove. The shaft 230 is inserted into the center bore hole to anchor the baseplate 200 to the glenoid cavity of the scapula. The peripheral ring portions 218 of the baseplate 200 may be press-fit into the circular groove to further anchor the baseplate 200 to the glenoid cavity. Next, one or more peripheral screws 150 may be used to further anchor the baseplate 200 to the glenoid cavity. The one or more screws 150 may be inserted through the one or more screw holes 210 into the glenoid cavity to secure the baseplate 200 to the bone.

Then, at least one defect surface may be reamed into a portion of the circular surface of the glenoid cavity bone within the defect directly under at least one screw hole 210. Alternatively, no reaming of a defect surface may be required if the defect provides a matching surface to the bottom surface of the augmentation member 160. After the at least one defect surface for the augmentation member 160 is prepared, at least one augmentation member 160 having the same or similar size to the at least one defect surface opening is selected. The selected augmentation member(s) 160 may be inserted through a screw hole 210 to contact or abut the bottom of the at least one defect surface providing support for the baseplate 200. The bottom of the augmentation device 160 does not penetrate the bottom of the at least one defect surface of the glenoid cavity bone. The threads 166 of the augmentation member 160 engage the threads 212 of the opening 210 of the plate 202 to secure the at least one augmentation member 160 to the plate 202. The at least one augmentation device 160 provides support to the baseplate 200 and allows for attachment to the glenoid cavity. Since the baseplate 200 includes multiple screw holes 210 that allow for insertion of separate augmentation members 160, the same baseplate 200 can be used for various bone deficiencies.

After the at least one augmentation member 160 is inserted into the baseplate 200, a glenosphere 110 may be selected and disposed over the baseplate 200. The glenosphere 110 may be positioned over and slid onto the locking screw 130. The through hole 122 of the glenosphere 110 receives the head 132 of the locking screw 130. The threads 136 of the head 132 of the locking screw 130 engages threads in the through hole 122 of the glenosphere 110 to couple the glenosphere 110 to the baseplate 200 by the locking screw 130.

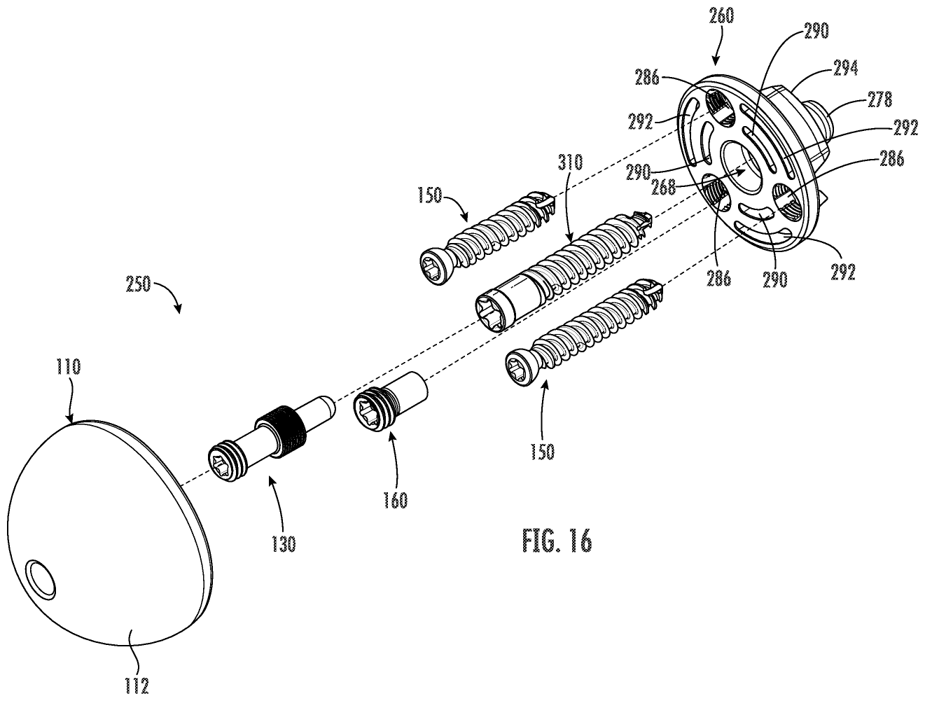

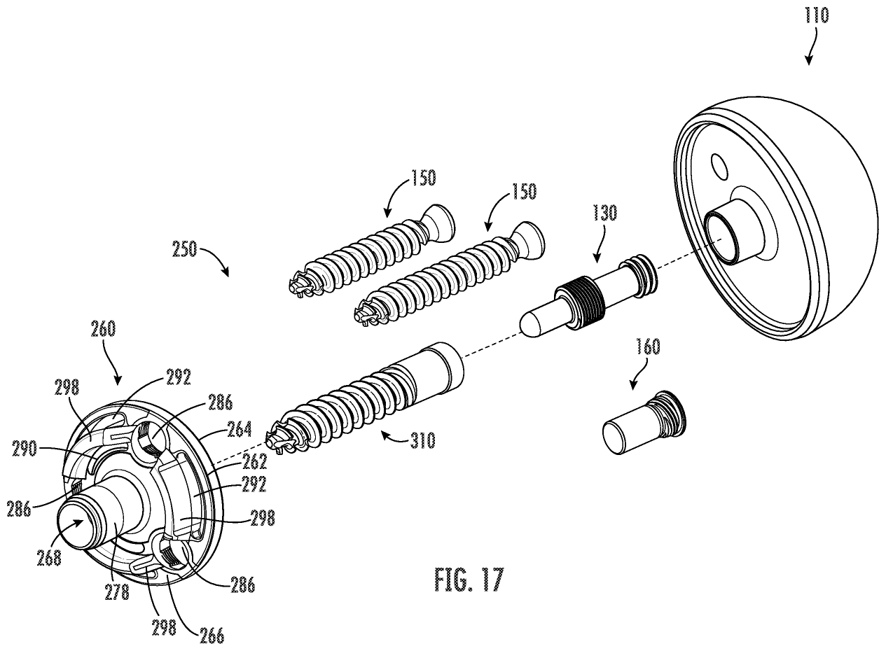

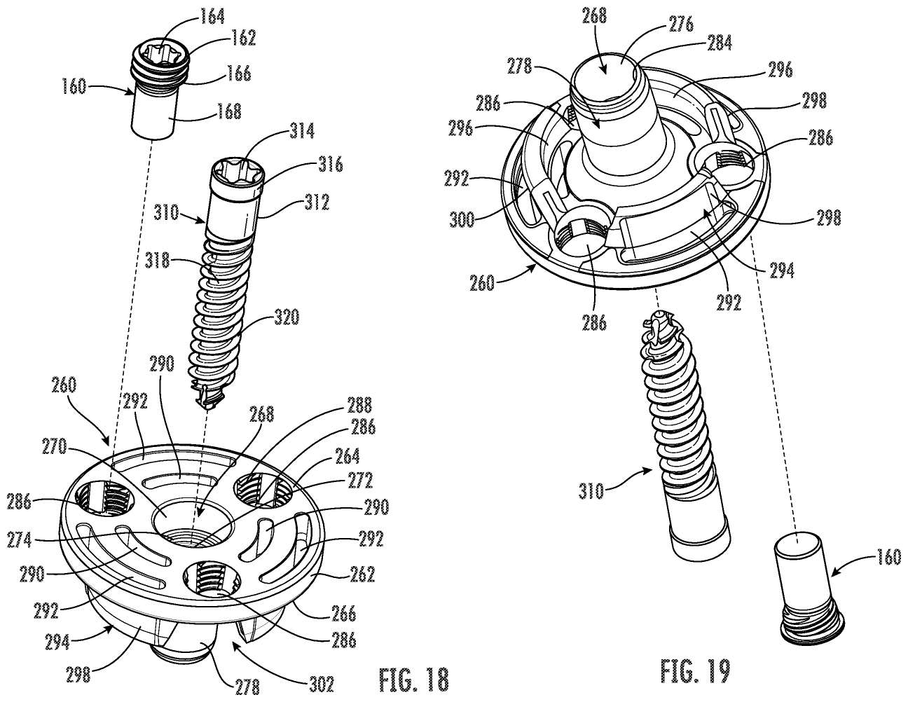

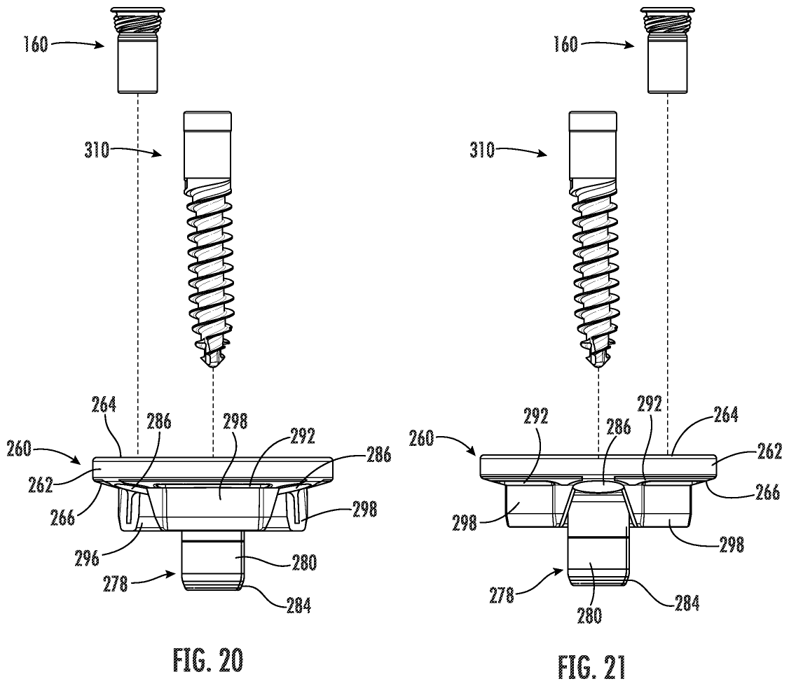

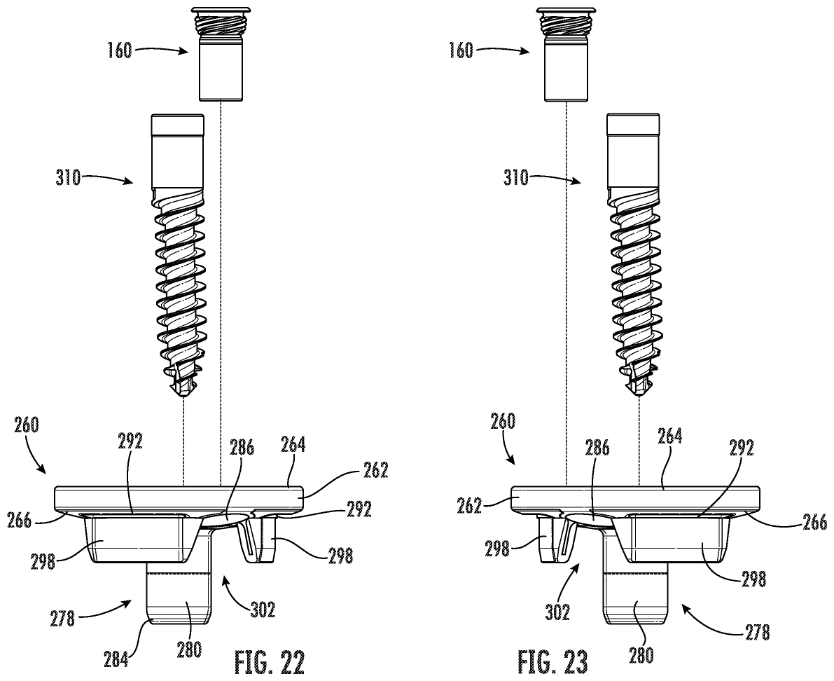

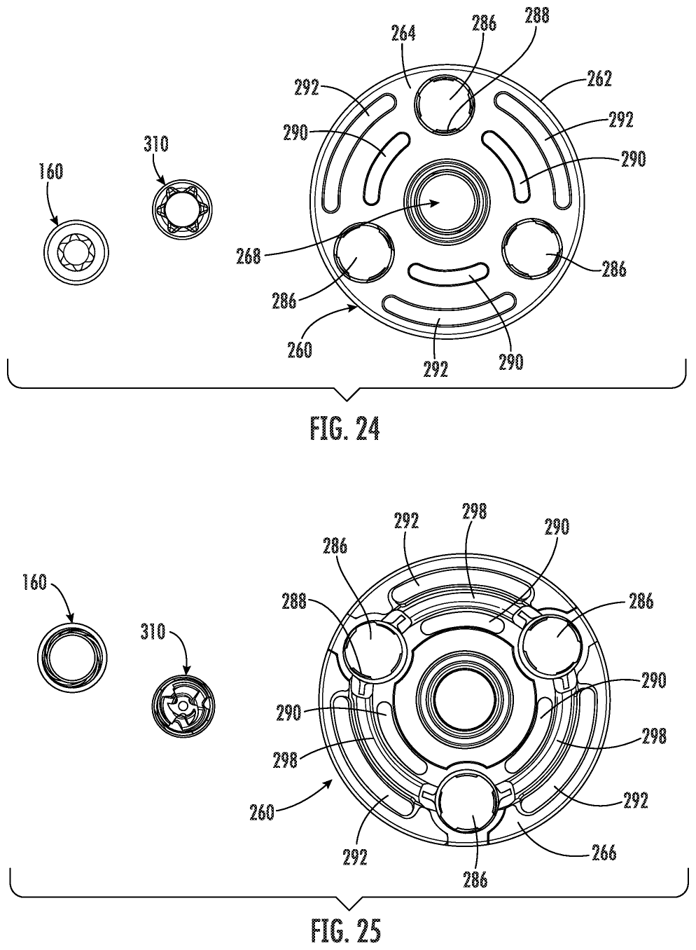

Referring now to FIGS. 16-25, there is illustrated another embodiment of a glenoid implant or reverse glenoid implant system 250. The implant system 250 includes a glenosphere 110, locking screw 130, at least one peripheral screw 150, at least one augmentation member 160, and a modular baseplate system with a baseplate 260 and screw 310. The locking screw 130, at least one peripheral screw 150, at least one augmentation member 160, and screw 310 are each received within the baseplate 260. The locking screw 130 couples the glenosphere 110 to the baseplate 260. The glenosphere 110, locking screw 130, at least one peripheral screw 150, at least one augmentation member 160 are as described in greater detail above, which will not be described again here for brevity sake.

The baseplate 260 may have a plate 262 and a shaft 278 coupled to and extending away from a bottom surface 266 of the baseplate 260, as shown in FIGS. 16-25. The plate 262 may have a top surface 264 and a bottom surface 266 opposite the top surface 264. The plate 262 may have, for example, a cylindrical shape with a circular top surface 264 and a circular bottom surface 266. The plate 262 may also include a central opening 268 extending from the top surface 264 through the plate 262 and through the shaft 278. The central opening 268 may be positioned, for example, in a central region of the plate 262. The central opening 268 may be, for example, configured or sized and shaped to receive a portion of the locking screw 130.

The central opening 268 may include a first portion 270, a second portion 272, and a third portion 276. The first portion 270 may extend from the top surface 264 of the plate 262 toward the bottom surface 266 of the baseplate 260. The second portion 272 may include threads 274. The threads 274 may be, for example, configured or sized and shaped to correspond to threads of the locking screw, as shown in FIGS. 16-17. The second portion 272 extends from the first portion 270 and into a portion of the shaft 278. The third portion 276 extends from the second portion 272 and further into the shaft 278. The central opening 268 extends through the baseplate 260 from the top surface 264 to the bottom of the shaft 278. The first portion 270 has a first diameter, the second portion 272 has a second diameter, and the third portion 276 has a third diameter. The first diameter may be larger than the second and third diameters. The second diameter may be larger than the third diameter. The third diameter may be smaller than the first and second diameters. The first portion 270 may be, for example, configured or sized and shaped to receive a portion of a glenosphere. The second portion 272 may be, for example, configured or sized and shaped to threadingly engage the threaded portion of the locking screw 130. The third portion 276 may be, for example, configured or sized and shaped to receive a post or screw 310 and a distal portion of the locking screw 130. The distal portion of the locking screw 130 may be received within the opening 314 of the screw 310.

The shaft or stem 278 includes an exterior surface 280, an interior surface 282, and a tapered edge 284 extending between the exterior surface 280 and the interior surface 282. The exterior surface 280 may have, for example, a porous structure on at least a portion of the length allowing for bone ingrowth. The porous structure may be, for example, 3D printed. The porous structure may be, for example, a porous metal structure or porous coating. The interior surface 282 of the shaft 278 or third portion 276 of the central opening 268 receives a portion of the head 312 of the screw 310.

The baseplate 260 may also include screw holes or holes 286, for example, three screw holes 286 equally spaced around the circumference of the plate 262. The screw holes 286 may extend through the plate 262 from the top surface 264 to the bottom surface 266. The screw holes 286 may have, for example, threads 288 along at least a portion of the interior surface of the screw holes 286. The screw holes 286 are configured or sized and shaped to receive a screw 150 for anchoring the baseplate 260 to a bone. The head 312 of the screw 150 may be seated within the screw hole 286 of the baseplate 260. The plate 262 may further include at least one relief or arcuate slot 290, 292 extending through the plate 262 from the top surface 264 to the bottom surface 266. The at least one relief 290, 292 may include at least one first or interior relief 290 and at least one second or exterior relief 292. The at least one first relief 290 may be, for example, three first reliefs 290 positioned circumferentially around the central opening 268 between the screw holes 286. The at least one second relief 292 may be, for example, three second reliefs 292 positioned circumferentially around the plate 262 between the screw holes 286. The first reliefs 290 are spaced apart from the second reliefs 292. The second reliefs 292 may be positioned near an exterior surface of the plate 262.

The plate 262 may include at least one peripheral ring portion 294 extending away from the bottom surface 266. The at least one peripheral ring portion 294 may be, for example, three peripheral ring portions 294 positioned circumferentially around the bottom surface 266. The peripheral ring portions 294 may be, for example, the same or different sizes. The peripheral ring portions 294 may be, for example, separated by a gap or space 302. The peripheral ring portions 294 may each include an interior surface 296 and an exterior surface 298. The interior surface 296 may be, for example, a uniform surface, while the exterior surface 298 may have, for example, threads 300 extending around the exterior surface 298. The interior and exterior surfaces 296, 298 may have, for example, a porous structure. The porous structure may be, for example, 3D printed. The at least one peripheral ring portion 294 may extend away from the bottom surface 266 of the plate 262 between the first reliefs 290 and the second reliefs 292. The peripheral ring portions 294 may be inserted to engage a prepared glenoid cavity. The peripheral ring portions 294 may be, for example, arcuate and follow the same profile as the first and second reliefs or slots 290, 292. The peripheral ring portions 294 may be, for example, positioned adjacent to the first and second slots 290, 292. In addition, the screw holes 286 in the plate 262 may be positioned between the peripheral ring portions 294 where the spaces 302 are positioned.

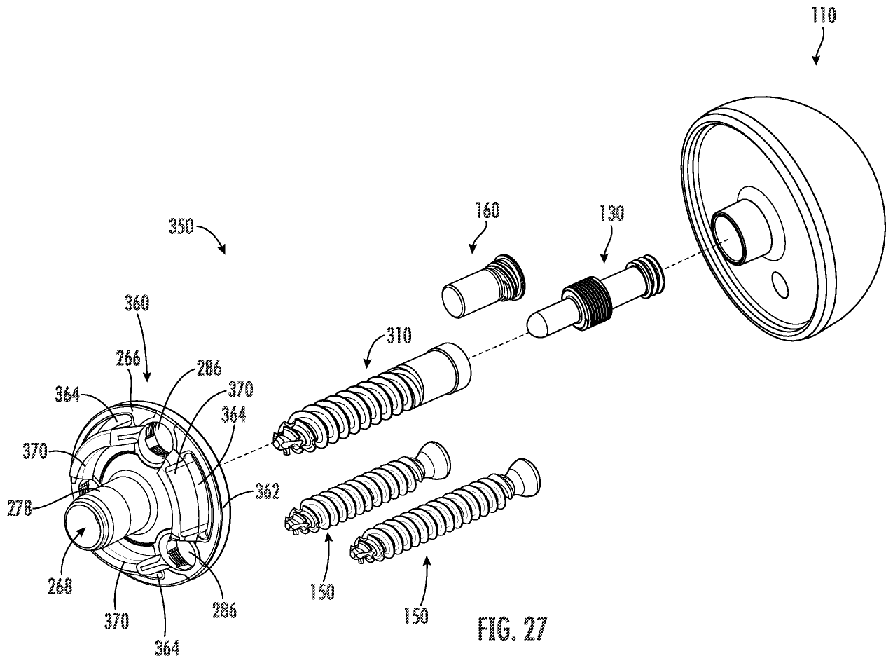

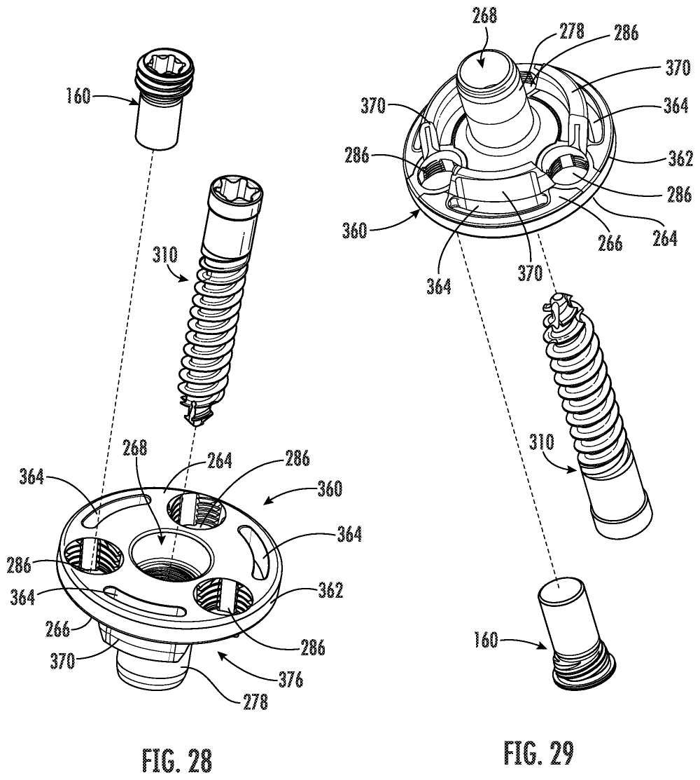

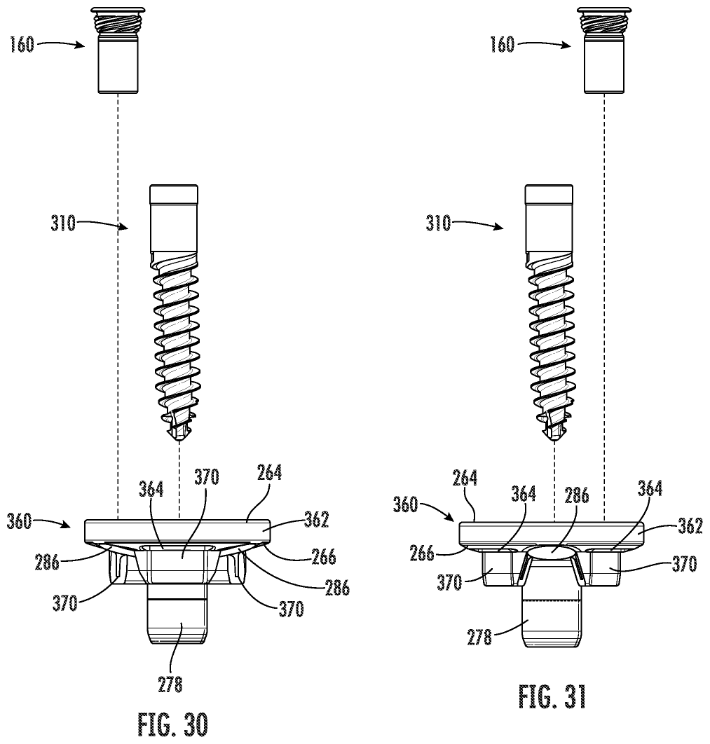

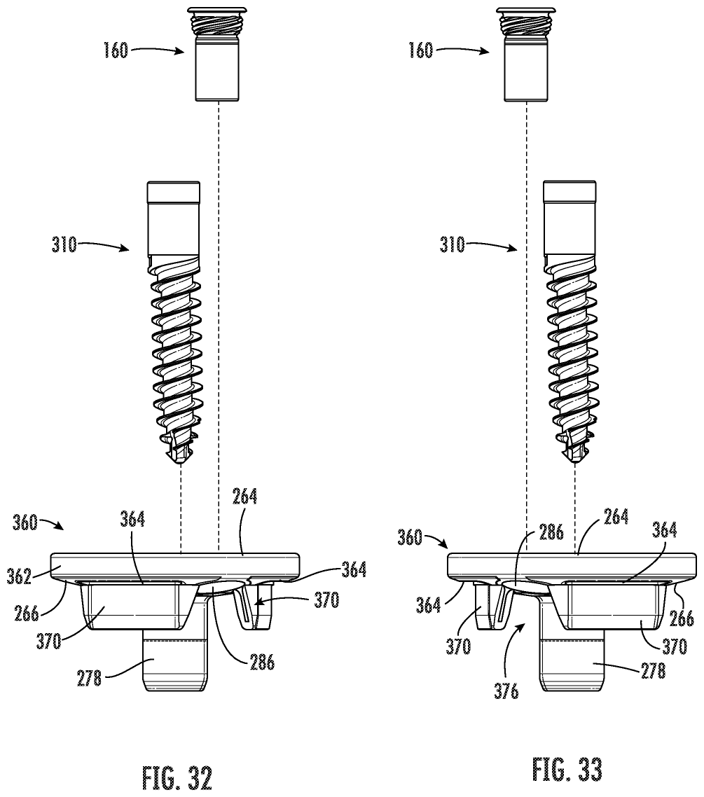

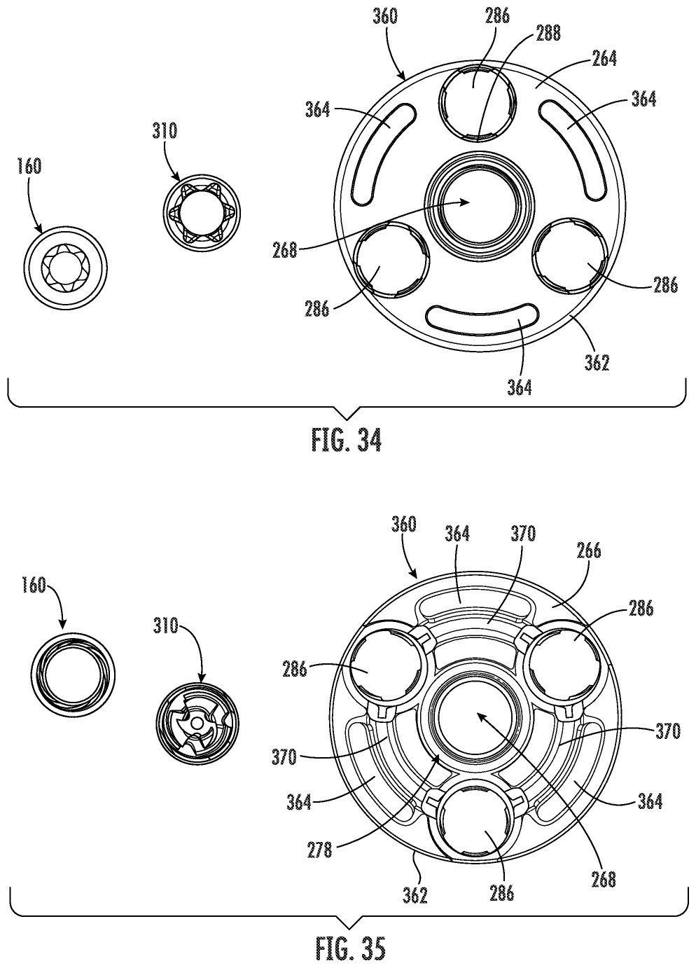

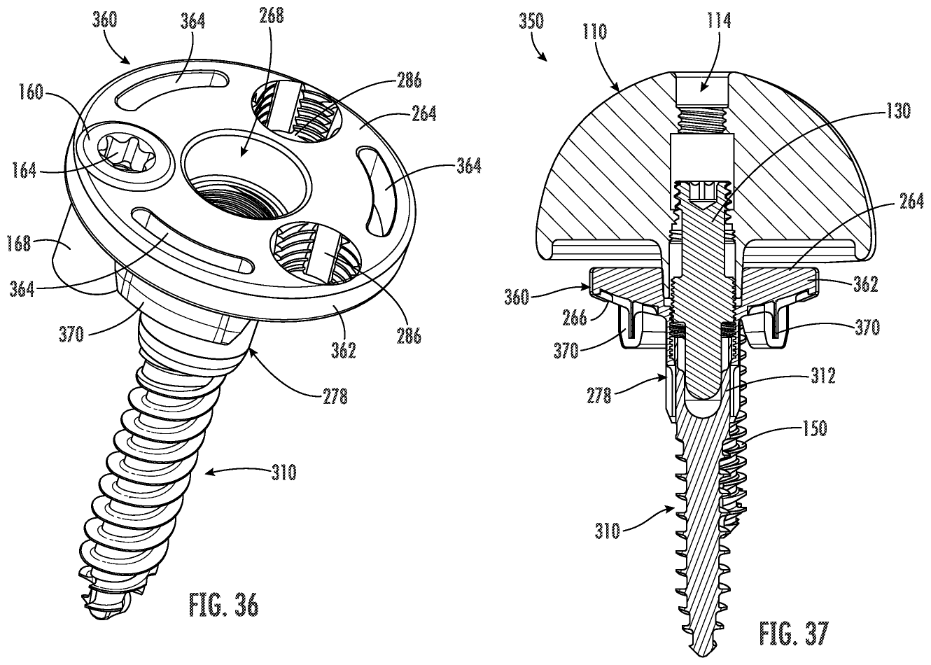

The screw 310 includes a head 312 and a stem 318 extending away from the head 312. The head 312 includes an opening 314 extending into the screw 310 from a top surface toward the stem 318 and a rim 316 extending circumferentially around a proximal or top end of the head 312. The opening 314 may include a drive feature for engaging a tool for insertion of the screw 310 through the baseplate 260. The head 312 may include a rim 316 extending around the top of the head 312. The stem 318 may have, for example, threads 320 extending along at least a portion of the length. The stem 318 may taper from the head 312 to the distal end of the screw 310. The screw 310 may be, for example, longer or shorter than illustrated in FIGS. 16-37 to provide the necessary fixation of the baseplates 260, 360 to the patient's bone.

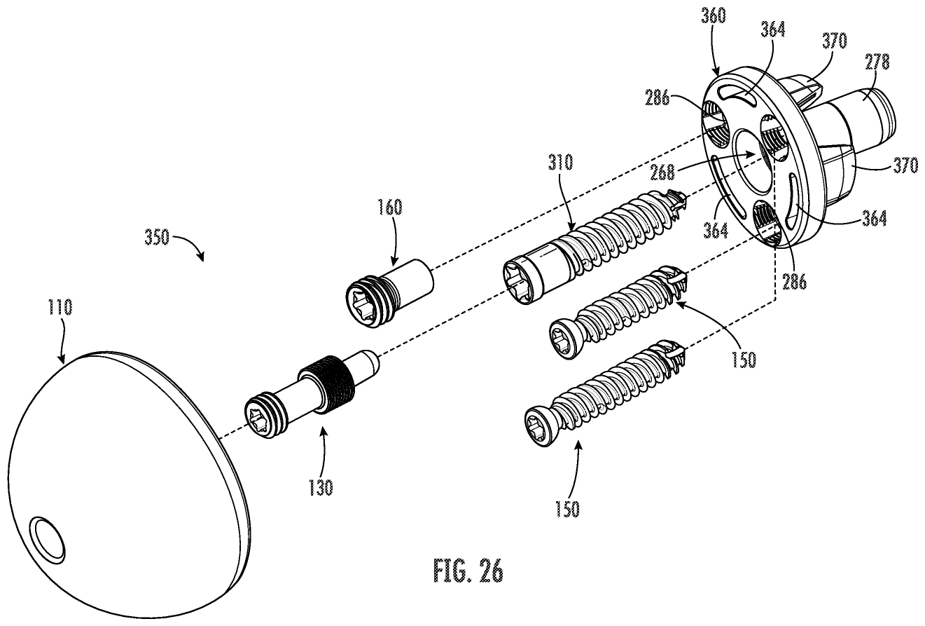

Referring now to FIGS. 26-37, there is illustrated another embodiment of a glenoid implant or reverse glenoid implant system 350. The implant system 350 includes a glenosphere 110, locking screw 130, at least one peripheral screw 150, at least one augmentation member 160, and a modular baseplate system with a baseplate 360 and screw 310. The locking screw 130, at least one peripheral screw 150, at least one augmentation member 160, and screw 310 are each received within the baseplate 360. The locking screw 130 couples the glenosphere 110 to the baseplate 360. The glenosphere 110, locking screw 130, at least one peripheral screw 150, at least one augmentation member 160, and screw 310 are as described in greater detail above, which will not be described again here for brevity sake.

The baseplate 360 may has a plate 362 and a shaft 278 coupled to and extending away from a bottom surface 266 of the baseplate 260, as shown in FIGS. 26-35. The plate 362 may have a top surface 264, a bottom surface 266, a central opening 268, and screw holes 286 as described in greater detail above with reference to plate 262, which will not be described again here for brevity sake.

The plate 362 may further include at least one relief or arcuate slot 364 extending through the plate 362 from the top surface 264 to the bottom surface 266. The at least one relief 364 may be, for example, three first reliefs 364 positioned circumferentially around the central opening 268 between the screw holes 286. The reliefs 364 may be positioned near an exterior surface of the plate 362.

The plate 362 may also include at least one peripheral ring portion 370 extending away from the bottom surface 266. The at least one peripheral ring portion 370 may be, for example, three peripheral ring portions 370 positioned circumferentially around the bottom surface 266. The peripheral ring portions 370 may be, for example, the same or different sizes. The peripheral ring portions 370 may be, for example, separated by a gap or space 376. The peripheral ring portions 370 may each include an interior surface 372 and an exterior surface 374. The interior surface 372 may be, for example, a uniform surface, while the exterior surface 374 may also have, for example, a uniform surface. The interior and exterior surfaces 372, 374 may have, for example, a porous structure. The porous structure may be, for example, 3D printed. The at least one peripheral ring portion 370 may extend away from the bottom surface 266 of the plate 362 adjacent to the reliefs 364. The peripheral ring portions 370 may be inserted to engage a prepared glenoid cavity. The peripheral ring portions 370 may be, for example, arcuate and follow the same profile as the reliefs or slots 364. The peripheral ring portions 370 may be, for example, positioned adjacent to the slots 364. In addition, the screw holes 286 in the plate 362 may be positioned between the peripheral ring portions 370 where the spaces 376 are positioned.

The surgical method for implanting the modular baseplates 260, 360 may be the same as described above with reference to plate 200, except for instead of inserting the plate 200 with a coupled threaded shaft 230, the modular baseplates 260, 360 may be inserted first and then the screw 310 inserted through the baseplates 260, 360 and into bone.

As may be recognized by those of ordinary skill in the art based on the teachings herein, numerous changes and modifications may be made to the above-described and other embodiments of the present disclosure without departing from the scope of the disclosure. The components of the implant systems, devices, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implant systems, devices, and/or systems may include more or fewer components or features than the embodiments as described and illustrated herein. For example, the components and features of implant system 100 may be used interchangeably and in alternative combinations as would be modified or altered by one of skill in the art. Further, the steps of the surgical methods associated with the implant system 100 may be used interchangeably and in alternative combinations as would be modified or altered by one of skill in the art. Accordingly, this detailed description of the currently-preferred embodiments is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

1. A glenoid implant for implantation into a glenoid of a patient during an orthopaedic surgical procedure, comprising:
a baseplate comprising a plate with a top surface and a bottom surface, the plate having: (i) a central opening extending into the plate from the top surface, and (ii) a plurality of screw holes extending from the top surface to the bottom surface of the plate, the plurality of screw holes being positioned circumferentially around the plate,
a shaft coupled to and extending downwardly away from a center of the bottom surface of the plate, the shaft having a number of threads formed in an outer surface thereof,
a first bone screw extending downwardly away from the bottom surface of the plate, the first bone screw having (i) a head positioned in a first screw hole of the plurality of screw holes, and (ii) an elongated threaded shank extending away from its threaded head,
a second bone screw extending downwardly away from the bottom surface of the plate, the second bone screw having (i) a head positioned in a second screw hole of the plurality of screw holes, and (ii) an elongated threaded shank extending away from its threaded head, and
an augmentation member extending downwardly away from the bottom surface of the plate, the augmentation member having (i) a threaded head positioned in a third screw hole of the plurality of screw holes, and (ii) an elongated non-threaded post extending away from its threaded head.

2. The glenoid implant of claim 1, wherein the elongated non-threaded post of the augmentation member has a porous structure disposed thereon.

3. The glenoid implant of claim 1, wherein the elongated non-threaded post of the augmentation member is shorter in length than the elongated threaded shanks of the first and second bone screws.

4. The glenoid implant of claim 1, wherein:
the plate of the baseplate further comprises a peripheral ring extending downwardly away from, and circumferentially around, the bottom surface of the plate,
the peripheral ring has an interior surface and an opposite exterior surface, and
the exterior surface of the peripheral ring has a number of threads formed therein.

5. The glenoid implant of claim 4, wherein the peripheral ring comprises a plurality of separate arcuate-shaped ring portions separated from one another by a gap.

6. The glenoid implant of claim 1, wherein the shaft is integral with the plate.

7. The glenoid implant of claim 1, further comprising a locking screw and a glenosphere, wherein:
the central opening of the plate has a number of threads formed therein,
the glenosphere has a number of threads formed therein, and
the locking screw has a first number of threads configured to engage the threads of the central opening and a second number of threads configured to engage the threads of the glenosphere.

8. A glenoid implant for implantation into a glenoid of a patient during an orthopaedic surgical procedure, comprising:
a baseplate comprising a plate with a top surface and a bottom surface, the plate having: (i) a central opening extending into the plate from the top surface, the central opening having a number of threads formed therein, and (ii) a plurality of screw holes extending from the top surface to the bottom surface of the plate, the plurality of screw holes being positioned circumferentially around the plate,
a shaft coupled to and extending downwardly away from a center of the bottom surface of the plate, the shaft having a number of threads formed in an outer surface thereof,
a pair of bone screws extending downwardly away from the bottom surface of the plate, each of the pair of bone screws having (i) a head positioned in one of the plurality of screw holes, and (ii) an elongated threaded shank extending away from its threaded head,
an augmentation member extending downwardly away from the bottom surface of the plate, the augmentation member having (i) a threaded head positioned in one of the plurality of screw holes, and (ii) an elongated non-threaded post extending away from its threaded head,
a glenosphere having a number of threads formed therein, and
a locking screw having (i) a first number of threads configured to engage the threads of the central opening and (ii) a second number of threads configured to engage the threads of the glenosphere.

9. The glenoid implant of claim 8, wherein the elongated non-threaded post of the augmentation member has a porous structure disposed thereon.

10. The glenoid implant of claim 8, wherein the elongated non-threaded post of the augmentation member is shorter in length than the elongated threaded shanks of the pair of bone screws.

11. The glenoid implant of claim 8, wherein:
the plate of the baseplate further comprises a peripheral ring extending downwardly away from, and circumferentially around, the bottom surface of the plate,
the peripheral ring has an interior surface and an opposite exterior surface, and
the exterior surface of the peripheral ring has a number of threads formed therein.

12. The glenoid implant of claim 11, wherein the peripheral ring comprises a plurality of separate arcuate-shaped ring portions separated from one another by a gap.

13. The glenoid implant of claim 8, wherein the shaft is integral with the plate.

14. A method of surgically implanting a glenoid implant into a glenoid of a patient's shoulder during performance of an orthopaedic surgical procedure, the method comprising:
surgically exposing an outer surface of the glenoid, the outer surface of the glenoid having a bone defect,
reaming the outer surface of the glenoid so as to form (i) a circular surface in the outer surface of the glenoid, (ii) a central bore hole that is concentric with the portion of the circular surface, and (iii) a circular groove that is concentric with the central bore hole,
inserting a baseplate of the glenoid implant into the central bore hole and circular groove, the baseplate having a plurality of screw holes,
aligning a first screw hole of the plurality of screw holes of the baseplate over the bone defect, and
inserting an augmentation member through the first screw hole such that a bottom surface of a non-threaded post of the augmentation member contacts a bottom surface of the bone defect.

15. The method of claim 14, further comprising:
inserting a first bone screw through a second screw hole of the plurality of screw holes of the baseplate such that a threaded shank of the first bone screw is threaded into the glenoid, and
inserting a second bone screw through a third screw hole of the plurality of screw holes of the baseplate such that a threaded shank of the second bone screw is threaded into the glenoid.

16. The method of claim 14, further comprising:
inserting a locking screw of the glenoid implant into a central opening in a top surface of the baseplate, and
coupling a glenosphere of the glenoid implant to the locking screw.

17. The method of claim 14, further comprising reaming the glenoid around the bone defect prior to insertion of the augmentation member.
